(19) United States
(12) Patent Application Publication
(10) Pub. No.: US 2007/0106161 A1
(43) Pub. Date: May 10, 2007

(54) Medical diagnostic imaging using visible light

(75) Inventors: Michael Eugene Werve, Modesto, CA (US); Mark Miles Hart, Livermore, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/256,574

(22) Filed: Oct. 19, 2005

(51) Int. Cl. A61B 6/00

(52) U.S. Cl. 600/476

(57) ABSTRACT

A system for medical diagnostic imaging using visible light comprises an emitting source for emitting light. The light is directed through a body structure and the light is viewed after it has passed through the body structure.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to imaging and more particularly to medical diagnostic imaging.

2. State of Technology

The article “History of Medical Diagnosis and Diagnostic Imaging,” Copyright © 1997-2005 Imaginis Corporation, www.imaginis.com provides the following state of technology information, “Radiology began as a medical sub-specialty in first decade of the 1900's after the discovery of x-rays by Professor Roentgen. The development of radiology grew at a good pace until World War II. Extensive use of x-ray imaging during the second world war, and the advent of the digital computer and new imaging modalities like ultrasound and magnetic resonance imaging have combined to create an explosion of diagnostic imaging techniques in the past 25 years.”

The article “The Electromagnetic Spectrum,” Imagine the Universe is a service of the High Energy Astrophysics Science Archive Research Center, http://imagine.gsfc.nasa.gov/docs/science/know_{—}12/emspectrum.html provides the following state of technology information: “visible—Electromagnetic radiation at wavelengths which the human eye can see. We perceive this radiation as colors ranging from red (longer wavelengths; ˜700 nanometers) to violet (shorter wavelengths; ˜400 nanometers.).”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for medical diagnostic imaging using visible light. The system comprises providing an emitting source for emitting light, directing the light through a body structure, and viewing the light after it has passed through the body structure. The light that is viewed after it has passed through the body structure is restricted to visible light. In one embodiment, the present invention provides an emitting source for emitting light, structure that directs the light through the body structure, and a view of the light after it has passed through the body structure. One embodiment of the invention includes a light filter for filtering the light positioned between the emitting source for emitting light and the body structure. Another embodiment of the invention includes a light filter for filtering the light positioned between the body structure and the view of the light after it has passed through the body structure.

The system of the present invention has many uses. For example, the system can be used for biological experimentation and medical diagnostics. One application of the system is the replacement of the use of x-rays in medical diagnostics for human and veterinary medicine. The system of the present invention can be used for passive diagnostic and active diagnostic. For example, the system of the present invention can be used for passive diagnostic for detection of broken bones, hematomas, aneurysms, restrictions, and other structural diagnostic. The system of the present invention can also be used for active diagnostic for bone setting, angioplasty, infrared pulse heating/cooling, etc.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates an embodiment of a system constructed in accordance with the present invention.

FIG. 2A illustrates the electromagnetic spectrum and visible light.

FIG. 2B illustrates visible light.

FIG. 3 illustrates another embodiment of a system constructed in accordance with the present invention.

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The Lawrence Livermore National Laboratory has been operated by the University of California continuously since its inception in 1952 and has extensive experience with optical phenomena. During the above ground nuclear testing in the 1950's and 1960's, laboratory personnel protecting their eyes at the time of detonation could clearly see the details of their bone structure. Detail included previously broken bones that had healed. It is now possible to produce cold light. This is light that does not contain any IR or UR frequencies. The present invention provides an obvious advantage for a doctor by using cold light for a real time internal view of a patient verses waiting for an X-ray room. No special room or shielding is required.

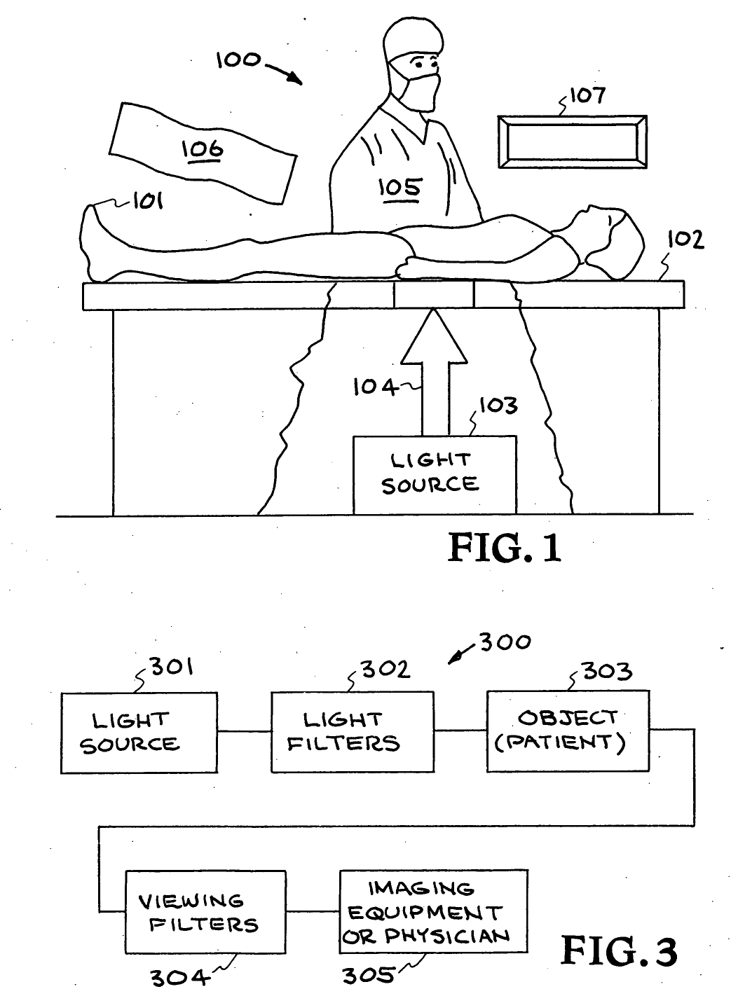

Referring now to FIG. 1, one embodiment of a system constructed in accordance with the present invention is illustrated. The system is designated generally by the reference numeral 100. The system 100 provides a system for imaging an individual's body structure 101. The system 100 includes an emitting source 103 for emitting radiation 104. A table or platform 102 is configured to support the body structure 101. The table or platform 102 is located adjacent the emitting source 103 and the body structure 101 is supported on the platform 102 and positioned to receive the radiation 104 emitted by the emitting source 103. An opaque blanket 106 is used to shield the individual and block excess light from around the area of inspection. The radiation 104 passes through the body structure 101 where it is viewed by the operator or physician 105.

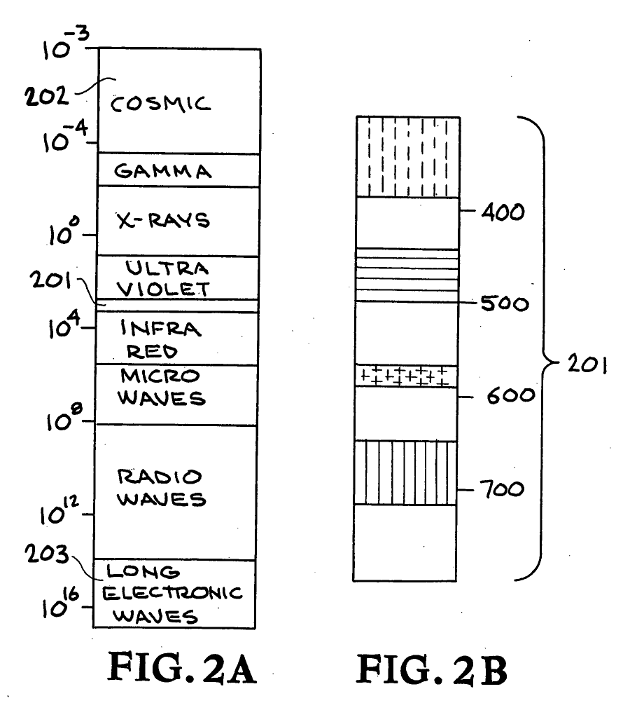

The emitting source for emitting radiation is an emitting source that emits high flux non-ionizing radiation. In one embodiment the emitting source for emitting radiation is an emitting source that emits cold visible light. The light spectrum can be broken in three parts: (1) long wave length infrared light (heat), (2) visible light, and (3) ultra violet. The electromagnetic spectrum is illustrated in FIG. 2A and visible light 201 is illustrated in FIG. 2B. As illustrated in FIG. 2A, the electromagnetic spectrum 200 extends from cosmic radiation 202 to long electric waves 203 and includes visible light 201. In one embodiment the emitting source 103 for emitting radiation 104 is an emitting source that emits light in the range of 400 to 700 nanometers wavelength.

The system 100 is a low cost, low risk system for imaging body structures, both soft and hard tissue. The system 100 has many uses. For example, the system 100 can be used for biological experimentation and medical diagnostics. One application of the system 100 is the replacement of the use of x-rays in medical diagnostics for human and veterinary medicine. Another use of the system 100 is a passive diagnostic, e.g., detection of broken bones, hematomas, aneurysms, restrictions, and other circulatory conditions. Another use of the system 100 is an active diagnostic, e.g., bone setting, angioplasty, infrared pulse heating/cooling. Referring again to FIG. 1, a broken bone can be set while the radiation 104 emitted by the emitting source 103 passes through the body structure 101 where it is viewed by the physician 105. This allows the physician 105 to see the bones move back into place. The physician 105 can observe the body structure 101 directly or the physician 105 can use a viewing filter 107 to observe the body structure 101. The viewing filter 107 can be used to enhance the view or to select contrast for the view.

Referring now to FIG. 3, another embodiment of a system constructed in accordance with the present invention is illustrated. This embodiment is designated generally by the reference numeral 300. The system 300 provides a system for imaging an individual's body structure. The system 300 includes the following structural elements: a light source 301 for emitting light, light filters 302, the object (patient) 303, viewing filters 304, and imaging equipment or physician 305.

The light source 301 emits high flux non-ionizing radiation. In one embodiment the emitting source 301 for emitting radiation is an emitting source that emits cold visible light. In one embodiment the emitting source 301 is an emitting source that emits light in the range of 400 to 700 nanometers wavelength. The light source 301 can be coherent or non-coherent. The light source 301 can produce a beam of light or it can be a scanning beam of light. The light filters 302 are used to eliminate undesired frequencies. The viewing filters 304 are used to enhance the view or to select contrast for the view.

The imaging equipment 305 includes digital imaging cameras, film imaging cameras, video imaging cameras, raster read, infrared imaging, visual imaging, and direct and electronic magnification. The system 300 also uses direct imaging by a physician through viewing filters.

A table or platform is configured to support the object (patient) 303. The table or platform is located adjacent the light source 301 and the object (patient) 303 is supported on the platform and positioned to receive the light. An opaque blanket is used to shield the individual and block excess light from around the area of inspection. The light passes through the body structure where it is viewed by the physician 305.

The system 300 is a low cost, low risk system for imaging body structures, both soft and hard tissue. X-rays can be harmful to the human body. X-rays are generated behind heavy shielding to prevent exposure to medical personnel. Most medical X-rays are produced on film that takes time to develop before a physician can use the information. The system 300 provides realtime inspections. No harmful rays leave the system so that medical personnel can stand next to the system indefinitely while it is in use. The system 300 can be used in the emergency room triage to screen patients for broken bones and other internal injuries. It can also be used in portable field diagnostic, in schools, in other locations where X-ray equipment cannot be used. Advantages include lower equipment costs, no need for licensing, inspection and specialized training.

The system 300 has many uses. For example, the system 300 can be used for biological experimentation and medical diagnostics. One application of the system 300 is the replacement of the use of x-rays in medical diagnostics for human and veterinary medicine. Another use of the system 300 is a passive diagnostic, e.g., detection of broken bones, hematomas, aneurysms, restrictions, and other circulatory conditions. Another use of the system 300 is an active diagnostic, e.g., bone setting, angioplasty, infrared pulse heating/cooling. Referring again to FIG. 3, a broken bone can be set while the radiation emitted by the emitting source 301 passes through the body structure 303 where it is viewed by the physician 305. This allows the physician 305 to see the bones move back into place.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. An apparatus for imaging a body structure, comprising:
an emitting source for emitting light,
structure that directs the light through the body structure, and
a view of the light after it has passed through the body structure.

2. The apparatus for imaging a body structure of claim 1 wherein said view of the light after it has passed through the body structure is a view of visible light.

3. The apparatus for imaging a body structure of claim 1 wherein said view of the light after it has passed through the body structure is a view of light in the range of 400 to 700 nanometers wavelength.

4. The apparatus for imaging a body structure of claim 1 including a light filter for filtering the light.

5. The apparatus for imaging a body structure of claim 1 including a light filter for filtering the light and wherein said light filter is positioned between said emitting source for emitting light and the body structure.

6. The apparatus for imaging a body structure of claim 1 including a light filter for filtering the light and wherein said light filter is positioned between the body structure and said view of the light after it has passed through the body structure.

7. The apparatus for imaging a body structure of claim 1 including imaging equipment that provides said view of the light after it has passed through the body structure.

8. The apparatus for imaging a body structure of claim 7 wherein said imaging equipment is a digital imaging camera or a film imaging camera or a video imaging camera or a raster read or an infrared imaging device or a visual imaging device or a direct magnification device or an electronic magnification device.

9. The apparatus for imaging a body structure of claim 7 wherein said emitting source for emitting light is a source for emitting coherent light.

10. The apparatus for imaging a body structure of claim 7 wherein said emitting source for emitting light is a source for emitting non-coherent light.

11. The apparatus for imaging a body structure of claim 7 wherein said emitting source for emitting light is a source for emitting a scanning beam of light.

12. The apparatus for imaging a body structure of claim 1 including a platform configured to support the body structure located adjacent said emitting source for emitting light.

13. A method of imaging a body structure, comprising the steps of:
providing an emitting source for emitting light,
directing the light through the body structure, and
viewing the light after it has passed through the body structure,
wherein said step of viewing the light after it has passed through the body structure comprises restricting said viewing to viewing visible light.

14. The method of imaging a body structure of claim 13 wherein said step of viewing the light after it has passed through the body structure comprises restricting said viewing to viewing light in the range of 400 to 700 nanometers wavelength.

15. The method of imaging a body structure of claim 13 including the step of filtering said light to eliminate undesired frequencies.

16. The method of imaging a body structure of claim 13 including the step of filtering said light to enhance the view.

17. The method of imaging a body structure of claim 13 including the step of filtering said light to select contrast for the view.

18. The method of imaging a body structure of claim 13 wherein said step of providing an emitting source for emitting light emits a scanning beam of light.

19. The method of imaging a body structure of claim 13 wherein said step of providing an emitting source for emitting light emits coherent light.

20. The method of imaging a body structure of claim 13 wherein said step of providing an emitting source for emitting light emits non-coherent light.
